(19) United States
(12) Patent Application Publication
(10) Pub. No.: US 2020/0022685 A1
(43) Pub. Date: Jan. 23, 2020

(54) Biopsate Recovery Device

(71) Applicant: Erbe Elektromedizin GmbH, Tuebingen (DE)

(72) Inventors: Achim Brodbeck, Metzingen (DE); Joerg Kronenthaler, Hirrlingen (DE); Lars Blobel, Ammerbuch-Entringen (DE); Thomas Staebler, Tuebingen (DE)

(21) Appl. No.: 16/512,927

(22) Filed: Jul. 16, 2019

(30) Foreign Application Priority Data
Jul. 17, 2018 (EP) 18183972.1
Jun. 7, 2019 (EP) 19179101.1

(51) Int. Cl. A61B 10/02; A61B 10/06; A61B 10/04

(52) U.S. Cl. CPC A61B 10/0266; A61B 18/02; A61B 10/04; A61B 10/06

(57) ABSTRACT

A biopsate recovery device (16) for recovering a biopsate (20) through an opening (46) with a recovery element (17) for fastening to or adjacent to a working tip (13) of a biopsy instrument (10) is disclosed, wherein the recovery element (17) is set up to—while the working tip (13) is being retracted through the opening (46)—be folded over the biopsate (20) in order to enclose the biopsate during the retraction of the working tip (13) through the opening (46) in order to separate the biopsate (20) from the environment during retraction through the opening (46). As a result of this, the biopsate (20) and/or the environment can be protected against contamination and/or cell transfer and/or germ transfer, and/or the biopsate (20) can be protected against being inadvertently stripped off at the opening (46).

### RELATED APPLICATION(S)

This application claims the benefit of European Patent Application No. 18183972.1, filed Jul. 17, 2018, and European Patent Application No. 19179101.1, filed Jun. 7, 2019, the contents of which are incorporated herein by reference as if fully rewritten herein.

### TECHNICAL FIELD

The invention relates to a biopsy instrument.

### BACKGROUND

From publication DE 10 2007 020 582 A1, a cryosurgical instrument and method for severing a tissue sample have been known, said instrument comprising a probe with a probe head for solid-freezing of a sample, as well as a support device, in which the probe is guided. By means of the support device, it is possible to support the surrounding tissue while the tissue is being severed. When the frozen-solid tissue sample is torn off, the probe head can be retracted into the support device so that, as a result, the tissue sample is safely accommodated in the support device.

From publication DE 10 2008 026 635 A1 a cryobiopsy probe has been known, which probe can be guided through a support hose. The support hose and the probe can be accelerated relative to each other in order to tear off the tissue sample with a defined tearing force.

During the flexible endoscopic tissue sample removal, (e.g., tissue sample from the pancreas) with the aid of a cryobiopsy probe through an organ wall (e.g., stomach wall), it is possible for parts of the sample or the entire sample to be lost on the entire recovery path or, specifically, while it is pulled through the organ wall. This may result in the transfer of germs or cross-contamination.

For the recovery of tissue samples it has been known to provide a recovery tube having a recovery section, wherein the probe with the tissue sample is pulled into the recovery region. The probe and the recovery tube are removed simultaneously. However, the recovery tube of the cryopuncture probe cannot be moved through the affected organ wall. The reason being that the recovery tube has too large a diameter and thus too great a force is required for moving the recovery tube forward. The risk of stripping the sample on the organ wall thus continues to exist. The tissue sample (e.g., tumor tissue) is pulled through healthy tissue (e.g., the stomach wall) before the sample has been retracted into the recovery tube. In doing so, tumor cells may be transferred. A loss of the tissue samples by stripping on the stomach wall, for example, may occur.

Furthermore, the punch biopsy has been known from prior art, wherein, by means of special types of needles (e.g., TruCut), a cylindrical biopsate can be harvested. However, the latter is frequently tainted with artifacts and the amount of biopsate is too small for an evaluation.

Furthermore, the fine needle biopsy has been known from prior art, wherein an injection needle is inserted into the tissue site from which a sample is to be removed and the cells are aspirated by the injection needle. Therefore, it cannot be used for harvesting larger biopsates.

The object of the invention is to state an improved concept for harvesting a biopsate.

### SUMMARY

This object is achieved with a biopsate recovery device and a biopsy instrument as described herein:

The biopsate recovery device according to the invention comprises a recovery element to be fastened to or adjacent to a working tip of a biopsy instrument (hereinafter also referred to as instrument). The working tip is disposed to hold the biopsate. The working tip may also be referred to as the head or probe head. The biopsate may adhere, for example, to the outside of the working tip, in particular be frozen thereto, and/or be received in the working tip. The working tip may be disposed to be guided through an opening, in particular in a tissue wall, to a removal site, for example an additional tissue wall behind said tissue wall. For example, the working tip may be disposed to be guided through a stomach wall in order to extract a biopsate from an organ, e.g., the pancreas, that is adjacent to the stomach. The working tip is disposed to be retracted through an opening, e.g., through an opening in a wall of tissue, an organ wall, or through an opening of a working channel of an endoscope or bronchoscope. The recovery element is disposed to be folded over the biopsate during the retraction of the working tip in order to enclose the biopsate during the retraction of the working tip through the opening, for example the organ wall, in order to separate the biopsate during the retraction through the opening from the environment—in particular transverse to the recovery direction, in particular from the wall around the opening. In particular, the biopsate can be held by means of the recovery element during the retraction of the biopsate through the opening, in particular through the opening of the tissue wall, separated from the edge of the opening or from the tissue wall or the tissue, i.e., at a distance from or separate from the edge of the opening or the tissue wall or the tissue, in order to not contaminate the biopsate, for example. Additionally or alternatively, the recovery device may be disposed to separate the tissue around the opening from the biopsate which may contain germs or degenerated cells, i.e., to hold the biopsate at a distance from or separate from the tissue in order to not transfer the germs or degenerated cells, for example.

While being retracted, the recovery element is preferably folded over from a first position (orientation) into a second position (orientation) in order to enclose the biopsate. Preferably, the recovery element has a first surface which, during recovery when the recovery element separates the biopsate, faces the biopsate and a second surface which, during recovery when the recovery element separates the biopsate, faces away from the biopsate. While folding over from the first position into the second position for enclosing the biopsate, the first surface, which is facing outward in the first position, faces inward due to being folded over in order to be facing the biopsate. Accordingly, the second surface is moved outward while being folded over.

By means of the recovery element it is possible to protect the biopsate and/or the tissue around the opening not only from a germ or cell transfer, but the biopsate can be protected, by means of the recovery element, against being mechanically stripped off while it is being pulled through the opening. This is in particular of importance for embodiments of the biopsy instruments according to the invention which are used for harvesting biopsates that have a transverse dimension, e.g., diameter, that is greater than the transverse dimension of the working tip and/or a puncture channel formed with the working tip in the target tissue and/or an opening made with the working tip in an organ wall.

Furthermore, a biopsy instrument is stated which comprises a biopsate recovery device according to the invention.

The biopsate recovery device and/or the biopsy instrument according to the invention may be developed further with one or more of the following features:

When the recovery element is folded over the biopsate in order to circumferentially enclose the biopsate, the biopsate may experience a mechanical protection from the recovery element, for example, against being stripped off the working tip. The recovery element may be disposed to circumferentially enclose the biopsate, in particular along the circumference of the shaft on which the working tip is arranged, along a circumference around the working tip and/or around the biopsate, in order to fluidically separate the biopsate from the tissue. When the biopsate is fluidically separated from the tissue by means of the recovery element in transverse direction, in particular perpendicularly to retracting direction, from the environment, the biopsate and the tissue can be particularly reliably protected against mutual contamination. The section of the recovery element that encloses the biopsate is preferably fluid-tight, in particular consist of a fluid-tight material. The section may be of plastic material, for example, silicone or polytetrafluorethylene. If the recovery element is disposed to form a fluid-tight shield or sheath for the biopsate, the recovery element can particularly effectively shield the biopsate against contamination due to fluid, in particular when the biopsate is being extracted through a tight opening in a tissue wall, which tissue wall would potentially directly contact the opening around the biopsate without recovery element during recovery.

The recovery element—in elastically undeformed state—may be disposed to enclose the biopsate at a distance transverse to the retracting direction or recovering direction or transverse to the longitudinal extension direction of the shaft and/or the working tip. The recovery chamber that the folded-over recovery element provides for the biopsate may have—in its elastically non deformed state transverse to the recovery direction—an inside diameter that is greater than the diameter of the biopsate.

Referring to the embodiments, a section of the recovery element for enclosing the biopsate may be resilient in such a manner that the section, when said section—while the working tip is pulled out of the body of the patient—is pressed against the biopsate and adapts to the form of the biopsate and remains in intimate contact therewith until the working tip is pulled out of the body.

The ratio of the diameter of the shaft—measured at a location at or in the recovery element—to the greatest outside diameter of the recovery element is preferably at most 1:1 to at least 1:6, particularly preferably at most 1:3 to at least 1:5. A ratio of 1:1 is possible if the greatest diameter of the working tip in its region for adhesion of biopsate is smaller than the diameter of the shaft.

The length of the section of the instrument which is disposed for receiving, in particular for the adhesion of, a biopsate and along which extends the part of the recovery element when enclosing the biopsate and thus separates the section, preferably corresponds to 2 to 20 times, in particular preferably to 4 to 10 times, the diameter of the shaft measured at a location at or in the recovery element.

While the folded-over recovery element preferably circumferentially (along a circumference around the biopsate) encloses the biopsate in such a manner that no fluid and/or aerosol and/or gas transverse to the recovery direction and/or radially to the working tip can reach the biopsate or move from the biopsate into the environment, the recovery element may be open in the direction counter the recovery direction, in particular in the direction in which the working tip is pointing.

The recovery element can completely enclose the biopsate, i.e. not only in circumferential direction, but a section of the recovery element folded around the biopsate may also be arranged in distal direction or in longitudinal extension direction of the working tip and/or the instrument between the distal end and the environment. When the biopsate is completely enclosed by the folded-over recovery element, the transfer of germs can be prevented in a particularly effective manner.

The recovery element may be provided with a structure that—when the working tip is being retracted—is disposed to come into engagement with the opening, the opening edge and/or the wall around the opening in order to aid the folding-over of the recovery element around the biopsate. During retraction, the structure comes into engagement with the wall, the opening edge and/or the opening wall and may temporarily be hung up there due to positive-locking or frictional locking, so that the folding-over of the recovery element is promoted by the retraction until the structured section of the recovery element is pulled through the opening. The structure may consist of a rough surface, for example; or the interior surface structure may comprise at least one structure element or several structure elements such as scales and/or nubs. A structure disposed for promoting the folding-over may be provided, for example, on one side of a part of the recovery element that faces outward during retraction of the recovery element when being folded over; and/or a specific structure for promoting the folding-over may be provided, for example, on one side of a part of the recovery element that is folded inward when the recovery element is being retracted.

The embodiments of the biopsate recovery device may comprise, for example, recovery elements that comprise a support element. Said support element is preferably elastically deformable transverse to the retracting direction and is disposed to support itself on the wall at the opening, preferably around the opening, during retraction in order to promote the folding-over. When guided through the opening in the direction to the site where the biopsate is to be removed, the support element is elastically deformed and, after passage through the opening, which can be expanded by passing the instrument, in particular the recovery element, is inclined—upon elimination of the outside force outside the opening—to assume again a greater diameter transverse to the opening direction or recovery direction (retracting direction). The support element that, due to elastic resetting forces, has again assumed a greater diameter—preferably a diameter greater than the opening—supports itself during the retraction against the wall that contains the opening, and thus remains temporarily hang up in order to promote the folding-over. Preferably, the support element supports itself around the opening during retraction.

During retraction, the support element preferably supports itself at least until the recovery element is folded over.

For example, the support element may be an elastically deformable ring. Preferably, the support element is arranged on the end of the recovery element that, during passage through the opening in the direction for receiving a biopsate with the working tip, is last to leave the opening.

The recovery element may consist of a material or have a coating, said material or coating being disposed to prevent an adhesion and/or freezing of the recovery element to the instrument, for example a cryobiopsy probe, in particular its working tip and/or its shaft, and/or to prevent a freezing and/or adhesion of the biopsate to the recovery element. For example, the recovery element may consist of a material or have a coating, said material or coating being disposed to prevent an adhesion and/or freezing of the recovery element to a biopsy instrument for harvesting a biopsate, said biopsy instrument being equipped with a biopsate recovery device according to the invention, and in particular prevent an adhesion and/or freezing to its working tip and/or its shaft.

When the recovery element is used with a cryobiopsy probe, it is possible, additionally or alternatively, for the recovery element to be preferably made of a material whose elasticity is not affected by the cooling of the working tip and/or the shaft. In particular, the elasticity of the part of the recovery element that is folded over, is preferably temperature-independent in those temperature ranges in which the part that can be folded over can assume a temperature, during the extraction of the biopsate by means of a cryobiopsy probe, in sections of the part.

The recovery element can comprise—in particular adjacent to or near a fastening location on the biopsy instrument, or adjacent to a fastening section of the recovery element that is affixed to the biopsy instrument—a subsection having an outside diameter that widens along a longitudinal section of the biopsy instrument. Between the subsection and the biopsy instrument in folded-over condition, the subsection can delimit a region of a recovery chamber. The outside diameter of the subsection can widen for moving the biopsy instrument through an opening in a wall in a direction that is counter to the direction (passage direction) in which the biopsy instrument is moved for passage, or narrow in passage direction in order to simplify moving through the opening. Preferably, the outside diameter of the subsection tapers—in the first position assumed by the recovery element in the direction of movement of the instrument toward the location where the biopsate is to be harvested, and/or the outside diameter of the subsection tapers—with the recovery element in the second, folded-over position—in the recovery direction, i.e., in the direction in which the biopsy instrument is removed from the location where the biopsate was taken for removal of the working tip with the biopsate. In this manner, the passage of the recovery element through the opening may be simplified. For example, the subsection may taper conically. Preferably the subsection is elastically deformable in radial direction and in a direction transverse to the recovery direction, respectively. In doing so, the passage of the recovery element through the opening in puncturing direction and/or the retraction of the recovery element through the opening through the opening may be particularly simplified.

The subsection which connects the section for enclosing the biopsate with the fastening section can have, in the transition to the fastening section of the recovery element, an outside diameter which, with respect to the outside diameter in the transition to the section for enclosing the biopsate, is in a ratio of at most 1:1 to at least 1:6; preferably at most 1:3 to at least 1:5, when the recovery element is not elastically deformed transversely to the longitudinal extension direction of the shaft. The stated ratio may refer to a position in which the free end of the recovery element points in the proximal direction and/or to a position in which the free end of the recovery element points in distal direction.

Preferably, the material or materials of the recovery element have a hardness of at least 20 to at most 80 Shore A, preferably at least 40 to at most 60 Shore A. In the exemplary embodiments, the recovery element may be composed of material which display different hardnesses, for example in Shore A. For example, the recovery element may be of a harder material in the fastening section than in the section (connecting section) of the recovery element adjacent in the direction toward the free end of the recovery element, which connects the section of the recovery element for enclosing the biopsate to the fastening section. Alternatively or additionally, the material of the connecting section may be harder than the material of the section of the recovery element for enclosing the biopsate. The difference in hardness between the materials may be, for example, at least 5 Shore A. For example, in the exemplary embodiments, the hardness of the sections may increase linearly, progressively or degressively from the section for enclosing being the least hard section, via the connecting section, to the fastening section being the hardest of the three sections.

The recovery element may have a opening up structure which comprises elongated elements and/or sections extending away from a base section or base element and being at a distance from each other in circumferential direction, which sections—due to the stiffness of the connection of the base section element or element with the elements from this path and/or section—attempt, when an outside force is removed, to resume their original orientation relative to the base section or base element and thus widen the mounting structure after, for example, the base element has been moved through a narrow opening into a wider region. The elongated elements or sections that are at a distance from each other in circumferential direction ensure that the subsection can be compressed radially in a controlled manner when the subsection is pushed through an opening in a tissue wall, and the connection with the base element or base section ensures that the subsection will particularly reliably open again due to the elastic resetting forces to assume its original form. For example, the recovery element many have elongated sections in the form of a wall thickness that increases and decreases in an alternating manner along the circumference and/or elongated reinforce element sections that may consist of another material than the subsection, e.g. of metal or another polymer.

When the recovery element in exemplary embodiments comprises, in longitudinal direction, adjacent different materials that form the sections of the recovery element, the sections may be configured with differing radial stiffnesses. In the exemplary embodiments, a connecting section may consist of a different material than a section for enclosing the biopsate. In doing so, exemplary embodiments of the recovery element according to the invention can be provided, wherein the connecting section displays another, e.g. greater, radial stiffness than the section for enclosing the biopsate.

Alternatively or additionally, in embodiments of the recovery elements, the greatest wall thickness of the section of the recovery element for enclosing the biopsate may be smaller than the greatest wall thickness of the connecting section of the recovery element that connects the section for enclosing the recovery element to the fastening section. In embodiments with a connecting section whose wall thickness alternately increases or decreases along the circumference, e.g. to form a mounting structure, the wall thickness—compared to the wall thickness of the section for enclosing the biopsate—can be measured at a location of the greatest wall thickness or at a location of the lowest wall thickness. Due to the wall thickness that continuously changes over sections of the recovery element in the direction toward the free end of the recovery element, in particular steadily and preferably monotonously (e.g., linearly) in the direction toward the free end of decreasing wall thickness, sections having a stiffness decreasing in the direction of the free end against radial deformation can be reliably provided which supports positioning variability and the ability of the recovery element to be reliably folded over the biopsate.

The wall of the recovery element preferably has a thickness of including 0.005 mm to 2 mm. Preferably, the wall has a thickness of <0.5 mm. The passage through an organ wall, in particular a stomach wall, is facilitated as a result of this.

When the recovery element is moved through the opening while the instrument is advanced to the location where the biopsate is to be extracted, the recovery element can be compressed and can be drawn lengthwise and in particular be expanded because the rear sections of the recovery element pass through the opening more slowly than the front sections which have already passed the opening due to the advance motion of the instrument. In doing so, preferably, the recovery element is elastically expanded at most to five times the unexpanded length, particularly preferably to at most 1.5 times the unexpanded length of the recovery element. The length is preferably measured in longitudinal extension direction of the biopsy element and/or the working tip. If this prevents an excessive lengthwise pulling of the retraction element when pulling or pushing through the recovery element through the stomach wall, for example, it is also possible to perform tissue biopsies directly behind the stomach wall, without having to dispense with enclosing the biopsate with the recovery element during the retraction through the opening.

Preferably, the recovery element has a first surface and/or a second surface to which cells and/or tissue will not adhere. The first surface and/or the second surface can display their non-adhesive (greatly inhibited adhesion) property, for example, due to micro-structuring and/or plasma treatment. Alternatively or additionally, the surface of the recovery element, for example the first surface and/or the second surface, may have an anti-adhesion coating against the adhesion of cells and/or tissue. For example, the surface may be coated with a fluoropolymer. For example, the surface—due to the coating and/or processing—may display a contact angle greater than or equal to 90°, particularly preferably greater than or equal to 110°. In doing so, it can be prevented that the cells are transferred by the recovery element.

The embodiments of the biopsy instruments according to the invention may be cryobiopsy probes, in particular. When the sections of the working tip of the cryobiopsy probe are cooled, tissue may freeze to it. A frozen biopsate may be harvested by being torn off. The low temperatures at the working tip can be achieved, for example, by utilizing the Joule-Thomson effect, as stated in prior art cited hereinabove.

Whereas the biopsy instrument preferably is a cryobiopsy probe, the biopsate recovery device according to the invention may also be used in other biopsy probes in order to separate a biopsate during removal from the environment in order to protect the environment and/or the biopsate and/or prevent a loss of the biopsate. For example, the biopsate recovery device can also be used on a biopsy probe with which a biopsate is punched out of tissue.

Preferably, the working tip is disposed for puncturing an organ wall with said working tip, i.e., the opening in the organ wall can be produced by means of the working tip.

For example, the recovery element can be manufactured by injection-molding around a working tip and/or a shaft of a biopsy instrument, in particular a cryobiopsy probe, and, in doing so, be connected to the biopsy instrument.

Preferred are instruments with at least one recovery element that can be interchanged in order to be able to dispose of the recovery element—in particular after the first use—and to be able to use the instrument with another recovery element.

For positioning the biopsy instrument for severing the sample from the tissue, the section of the recovery element for enclosing the biopsate can be received in a section of the recovery element, in particular, inserted, rolled in and/or folded in, said section connecting the section for enclosing the biopsate with the biopsy instrument. In one state of the embodiments of the instrument according to the invention, the section of the recovery element for enclosing the biopsate is received in a section of the recovery element, in particular inserted, rolled in and/or folded in, said section connecting the section for enclosing the biopsate with the biopsy instrument.

The biopsy instrument may comprise a securing element which is disposed to prevent an unwanted folding-over during the positioning of the working tip for severing the sample. The securing element can be deactivated before the retraction of the biopsy instrument for removal of the sample, so that a folding-over is possible during retraction.

### BRIEF DESCRIPTION OF THE DRAWINGS

Additional advantageous features with which the recovery device according to the invention or the biopsy instrument according to the invention can be further developed, or preferred embodiments of the recovery device according to the invention or the biopsy instrument according to the invention can be inferred from the description and the figures hereinafter. They show schematically and in an exemplary manner:

FIG. 1a—an exemplary embodiment of a biopsy instrument according to the invention, in a sectional view with an exemplary recovery device according to the invention in a first orientation;

FIG. 1b—the exemplary embodiment of the biopsy instrument according to the invention as in FIG. 1a, in a sectional view with the recovery device in a second orientation;

FIGS. 2a to 2i—an exemplary embodiment of a biopsy instrument according to the invention, for examples the instrument according to FIG. 1, while a biopsate is being taken;

FIG. 2i′—a modification of the exemplary embodiment of a biopsy instrument according to the invention as in FIGS. 2a to 2i;

FIGS. 3a and 3b—an exemplary embodiment according to an alternative embodiment of a recovery element according to the invention;

FIGS. 3c and 3d—a modification of the exemplary embodiment as in FIGS. 3a and 3b; and

FIGS. 4-12—additional exemplary embodiments of biopsy instruments or recovery elements according to the invention.

### DETAILED DESCRIPTION

The instrument according to the invention is an instrument for harvesting a biopsate. Harvesting of a biopsate by means of the instrument according to the invention may involve the separation by punching, the aspiration of tissue or the cutting-off and holding with a clamp. However, preferably, the biopsy instrument is a cryobiopsy instrument or a cryobiopsy probe. In the case of these, the working tip of the instrument can be cooled, for example by utilizing the Joule-Thomson effect inside the working tip, as has been amply disclosed by prior art. The working tip is pressed against the tissue at the site where the biopsate is to be removed, or pressed or punctured into the tissue. By strongly cooling the working tip, the tissue adjacent the working tip can be frozen solid. By removing the working tip from the site where the tissue has frozen to the working tip, in particular by an abrupt movement, the frozen tissue can be torn out of the tissue bond and be recovered for examination.

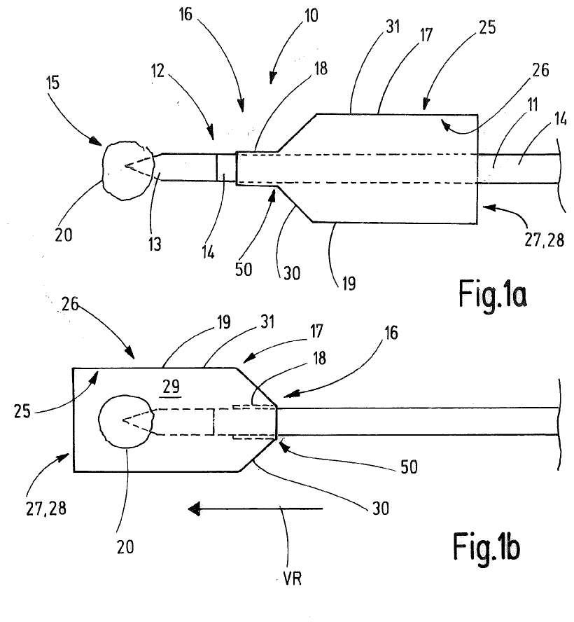

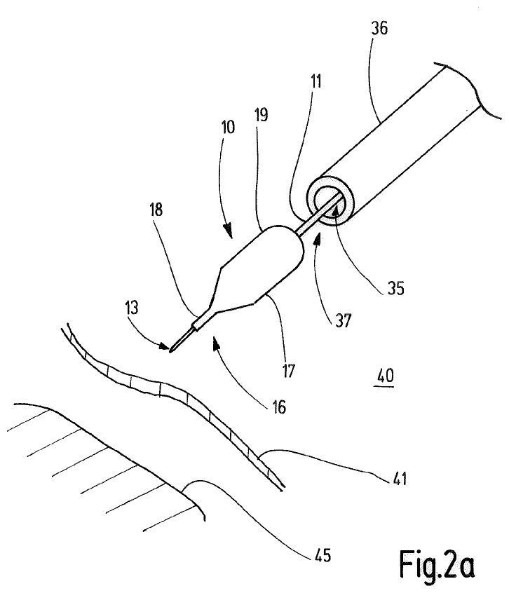

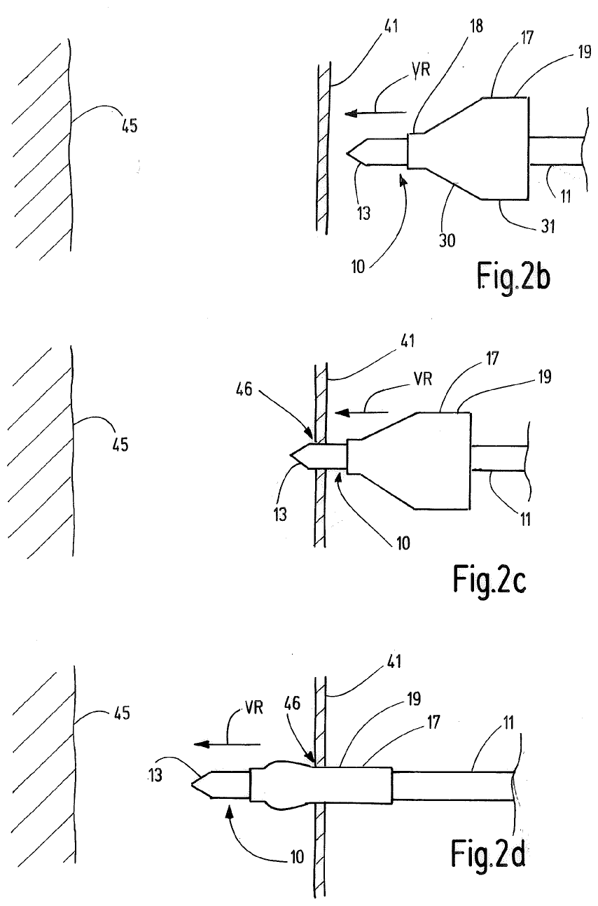

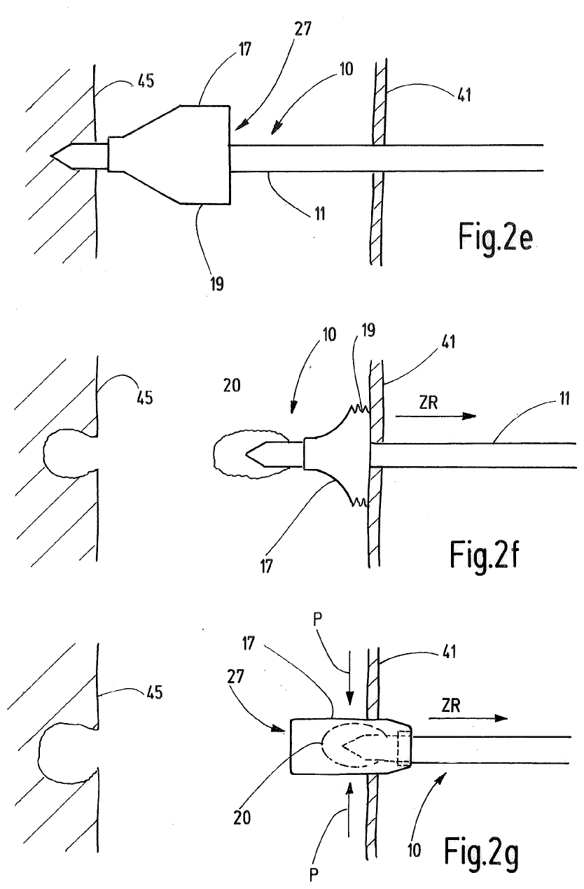

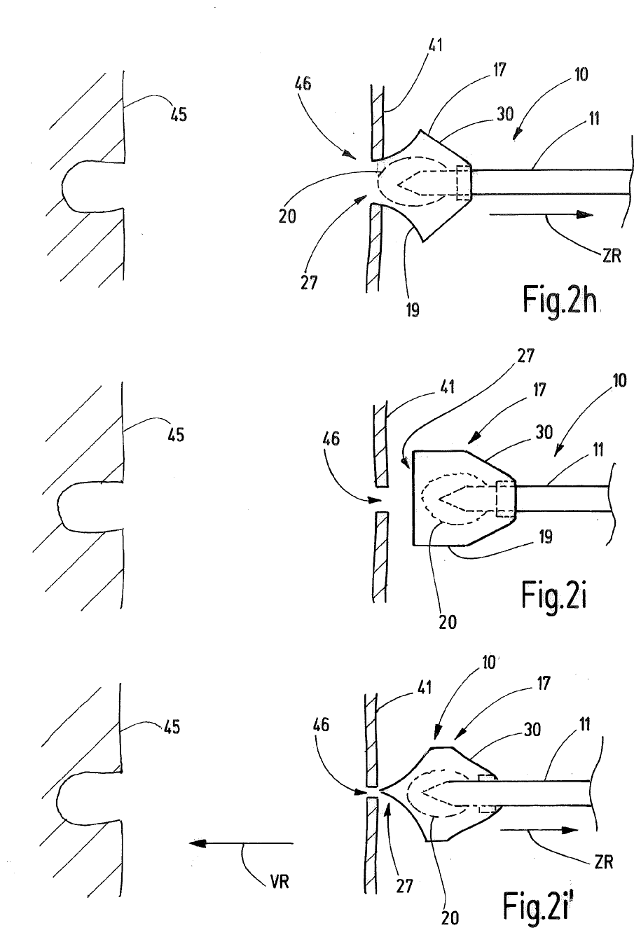

Exemplary embodiments of biopsy instruments 10 according to the invention, as are depicted, for example, by FIG. 1a, preferably comprise an elongated flexible shaft, whose end 12 is provided with a working tip 13. As shown, the shaft 11 may be jacketed in heat-insulating plastic material 14, while the working tip 13 preferably consists of bare metal in order to attain good heat transfer from the tissue to the working tip 13. The working tip 13 is preferably disposed to puncture—with the working tip 13—a tissue or organ wall, for example, the stomach wall 41 (see, for example, FIG. 2a). The biopsy instrument 10 is provided with an exemplary embodiment of a biopsate recovery device 16 according to the invention. Adjacent to the distal end 15 of the instrument 10, in particular adjacent to the working tip 13, there is provided a recovery element 17 around the shaft 11 and/or the working tip 13, said recovery element 17, for example, consisting of plastic material, for example of PTFE or silicone. The recovery element 17 has a fastening section 18, via which the recovery element 17 is fastened to the shaft 11 adjacent to the working tip 13 and/or to the working tip 13. Preferably, the fastening section 18 is fixed in place so as to not be slidable relative to the shaft 11 and/or the working tip 13 along the shaft 11 and/or the working tip 13. A part 19 of the recovery element 17 adjoins the fastening section 18 in order to enclose the biopsate 20 which can be frozen solid to the working tip 13 so as to hold the biopsate 20 for recovery.

In FIG. 1, the part 19 for enclosing the biopsate 20 is shown in a position or orientation in which a first surface 25 (the outside in this orientation) of the part 19 is oriented outward, and a second surface 26 (the inside in this orientation) is oriented inward. The open, free end 27 of the part 19 for enclosing the biopsate 20 is oriented in this position in a direction that is counter the direction into which the working tip 13 is pointing.

The part 19 for enclosing the biopsate 20 can be folded over from this position or orientation into a second position or orientation. In doing so, the open, free end 27 of the recovery element 17 is moved around the shaft 11 and the working tip 13 beyond the fastening section 18 in the direction of the working tip 13 or the distal end 15 of the instrument 10. In doing so, the fastening section 18 remains preferably fixed in place, so as to be immovable relative to the shaft 11 and/or the working tip 13 on the shaft 11 and/or the working tip 13. During the movement of the end 27 and the folding-over of the part 19 of the recovery element 17, the second surface 26, which when not folded over faced inward, is at least partially oriented outward, and the first surface 25, which was faced outward when not folded over, is at least partially oriented inward, in order to be facing the shaft 11 and/or the working tip 13 and/or the biopsate 20. FIG. 1b shows the instrument with the recovery element 17 in folded-over orientation. The free end 27 of the recovery element—as folded over—or the opening 27 on the free end 27 of the part 19 for enclosing the biopsate 20—as folded over, is oriented in this position in the direction in which the working tip 13 does point. The part 19 of the recovery element 17 for enclosing the biopsate 20 has such a length that the end 27 of the folded-over recovery element 17 extends beyond the biopsate 20. The circumferential enclosure of the biopsate 20 by the folded-over recovery element 17 encloses a recovery chamber 29, which—with the not elastically deformed recovery element 17—has preferably a greater inside diameter D1 than the biopsate 20. Preferably, the folded-over recovery element 17 encloses the biopsate 20 around the biopsate 20 at a radial distance between the recovery element and the biopsate 20.

The subsection 30 of the part 19 of the recovery element 17 for enclosing the biopsate 20 adjoining the fastening section 18 may be elastically deformable (transversely relative to the shaft 11 and/or relative to the working tip 13 and/or the biopsate 20) in radial direction. The subsection 30 may have the form of a cone or be conical. An adjoining additional subsection 31 for enclosing the biopsate 20—with the recovery element 17 folded-over, said subsection being arranged between the free end 27 and the subsection 30—can be deformed but not elastically deformed. Whereas the elastically deformable subsection attempts to return into its original form upon the removal of a deforming force, this cannot or only to a minimal decree apply to the non-elastically deformable additional subsection 31. The additional subsection 31 adjoining the elastically deformable subsection 30, however, may also be elastically deformable.

FIG. 2a shows an exemplary embodiment of an instrument 10 according to the invention, for example the instrument 10 according to the invention as described in conjunction with FIGS. 1a and 1b, whose shaft 11 is guided through a working channel 35 of an endoscope 36 and whose working tip 13 and recovery element 17 are arranged in front of the distal end 37 of the endoscope 36. The endoscope 36 is inserted through the esophagus into the stomach 40 of the patient and is arranged with the working tip 13 in front of the stomach wall 41 of the patient. The objective is to extract a biopsate 20 from the pancreas 45 of the patient by means of the instrument 10, the pancreas being next to the stomach 40.

To do so, as depicted by FIG. 2b, the instrument 10 is moved forward in puncturing or advance direction VR in the direction of the stomach wall 41, and the stomach wall 41 is punctured by means of the working tip, i.e., an opening 46 is made in the stomach wall 41 as shown by FIG. 2c.

The instrument 10 is further advanced through the thusly made opening 46 in the stomach wall 41, in which case also the part 19 of the recovery element for enclosing the biopsate 20 is pushed forward through the opening 46. When the recovery element 17 passes through the opening 46, it is radially squeezed together. The wall of the recovery element 17, in particular the wall 19 for enclosing the biopsate 20, has a thickness including 0.005 mm to including 2 mm. The wall, in particular the wall of the part 19 for enclosing the biopsate 20 preferably has a thickness of <0.5 mm. In particular when the maximum thickness of the wall of the recovery element 17 or at least the maximum thickness of the part 19 that encloses the biopsate 20 is between including 0.005 mm to including 2 mm or smaller, the passage through an organ wall—like in this example the stomach wall 41—is facilitated as a result of this.

When, as also in the embodiment according to FIGS. 1a, 1b, a subsection 30 adjoining the fastening section 18 for enclosing the biopsate 20 is configured so as to taper in advance direction VR, in particular in a conically tapering manner, it may be easier to push the recovery element 17 through the opening 46 in the stomach wall 41.

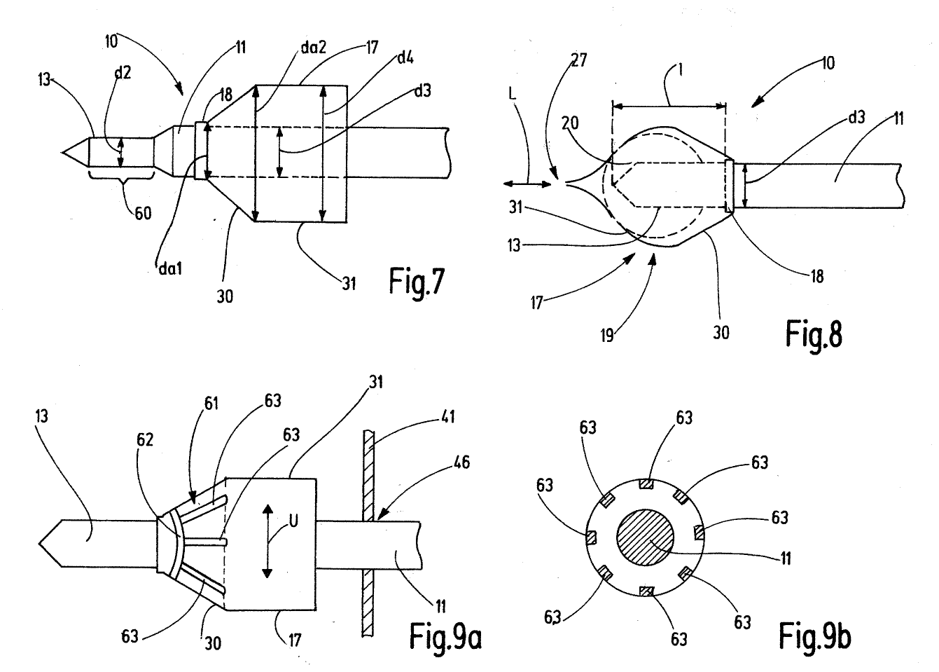

The subsection 30 that connects the section 31 for enclosing the biopsate 20 to the fastening section 18 can comprise—at the transition to the fastening section 18 of the recovery element 17 an outside diameter da1 (see, for example, FIG. 7) which is at a ratio to the outside diameter da2 at the transition to the section 31 for enclosing the biopsate 20, of at most 1:1 to at least 1:6, preferably at most 1:3 to at least 1:5, when the recovery element 17 is not elastically deformed transversely with respect to the longitudinal extension direction of the shaft 11. The stated ratio may relate to a position in which the free end 27 of the recovery element 17 points in proximal direction and/or to a position in which the free end 27 of the recovery element 17 points in distal direction.

Preferably, the subsection 30 is not directly connected by means of the shaft 11 or the working tip 13 to the instrument 10, but via the fastening section 18. The subsection 30 is radially elastically deformable or transversely with respect to the retracting direction ZR and/or the puncturing direction VR. When the subsection 30 between the stomach wall and the pancreas has moved out through the opening 46, said subsection may again assume its original form due to its elasticity.

When the recovery element 17, while the instrument 10 is being advanced to the site on the pancreas 45 where the biopsate 20 is to be harvested, is moved through the opening 46, the recovery element 17—as it is being pulled through or pushed through—can be pulled lengthwise, and in particular elongated, because the rear sections of the part 19 of the recovery element 17 move through the opening 46 more slowly than the front sections that have already passed through the opening 46. Preferably, the recovery element 17 and/or a part 19 of the recovery element that adjoins the fastening section 18 is elastically extended at most up to five times the non-extended length, particularly preferably to at most 1.5 times of the non-elongated length of the recovery element 17 or the part 19. The length is preferably measured in longitudinal extension direction of the biopsy instrument 10 and/or the working tip 13. If, due to that, an excessive lengthwise elongation of the recovery element 17 is prevented while, for example, the stomach wall 14 is being punctured, it is also possible to obtain tissue biopsies directly behind the stomach wall 14, without having to dispense with the enclosing of the biopsate 20 by the recovery element 17 during the retraction through the opening 46.

The instrument 10 is pushed further through the opening 46 and when the recovery element 17 has left the opening 46 between the stomach wall 41 and the pancreas 45, the elastic sections 30—to the extent that they exist—of the recovery element 17 can again assume the original form due to radial expansion. The instrument 10 is moved forward until the working tip 13 presses against the tissue of the pancreas 45 or punctures the tissue of the pancreas 45 (FIG. 2e).

The working tip 13 is cooled and, as a result of this, the tissue is frozen to the working tip 13. The tissue is released from the pancreas 45 by abrupt retraction of the instrument 10 in recovery direction ZR (arrow in FIG. 2f).

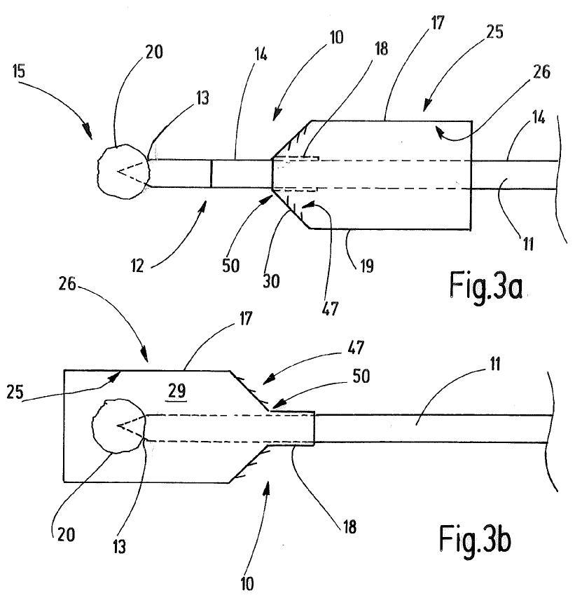

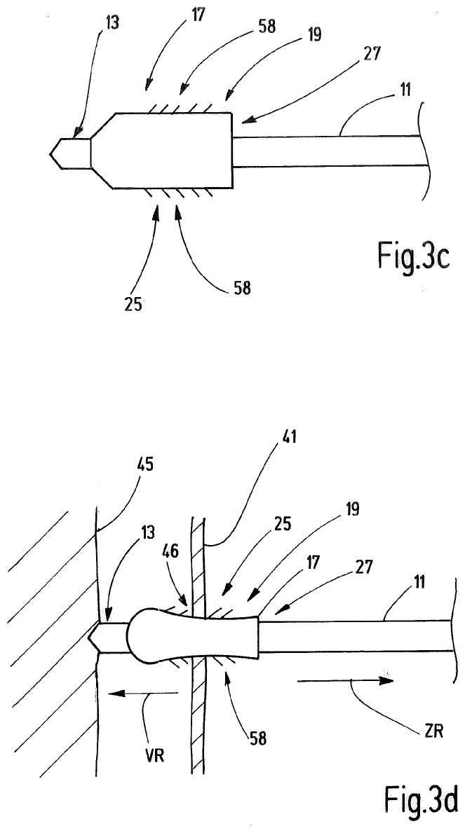

The working tip 13 is pulled out through the opening 46 from the intermediate space between the pancreas 45 and the stomach wall 41. In doing so, the recovery element 17 first comes into abutment and/or engagement with the stomach wall 41 and, by the retracting movement of the shaft 11 and abutment against the stomach wall 41 or the engagement with the stomach wall, is folded over the working tip 13 or over the biopsate 20 in order to—as illustrated by the arrows P in FIG. 2g—shield the latter transversely with respect to recovery direction ZR, in particular while pulling through the stomach wall 41 (see, in particular, FIG. 2h). The recovery element 17 may be provided with a structure 47 (see, for example, FIG. 3b) that is disposed to come into engagement with the edge of the opening 46 or with the stomach wall 41 around the opening 46 while the working tip 13 is being retracted in order to aid the folding-over of the recovery element 17 around the biopsate 20. The structure may be, for example, a rough surface, and/or the surface structure may comprise scales and/or nubs, so that the structure comes into engagement with the stomach wall 41 of the edge of the opening 46 and becomes hung up there, so that the recovery element 17 is folded over due to the retraction. Pulling the recovery element 17 through the opening 46 in the stomach wall 41 while folding over the part 19 of the recovery element 17 for enclosing the biopsate 20 may be facilitated when the not folded-over subsection 30 of the part 19 adjoining the fastening section is tapered outward for enclosing the biopsate 20 in recovery direction ZR (retracting direction) when the outside diameter decreases conically, for example in the recovery and retracting direction ZR.

The recovery element 17 may be disposed to enclose the biopsate 20 circumferentially along a circumference around the working tip 13 in a closed manner in order to fluidically separate the biopsate 20 from the tissue, in particular the stomach wall tissue, in radial direction. When the biopsate 20 is fluidically separated from the tissue by means of the retraction element 17 transversely—in particular perpendicularly to the retracting direction (RZ)—from the environment, the biopsate 20 and the tissue can be particularly reliably protected against mutual contamination. The section of the recovery element that encloses the biopsate, accordingly, is preferably fluid-tight in particular of fluid-tight material. When the recovery element 17 forms a fluid-tight sheath or shield transversely to the retracting direction ZR, this can shield the biopsate 20, in particular while being pulled through the opening 46 (FIGS. 2g, 2h) in a direction transverse to the longitudinal axis of the instrument 10, against contamination by fluids. While the folded-over retraction element preferably circumferentially encloses the biopsate so that no fluid and/or aerosol and/or gas—in a direction transverse to the retracting direction and/or radially with respect to the working tip, and/or have the shaft—will reach the biopsate or move from the biopsate into the environment, the retraction element is open—in the exemplary embodiments—in the direction counter the recovery direction, in particular the direction, in which the working tip is pointing.

FIG. 2h shows how the recovery element is squeezed together when being pulled through the opening. In doing so, the retraction element folded over the biopsate helps the biopsate while being pulled through the wall to not become hung up on the opening edge, so that the biopsate, while being pulled through the organ wall, is not stripped off the working tip. Preferably, the recovery element consists of a material or has a coating that is disposed to prevent an adhesion and/or freezing of the retraction element to the instrument, for example a cryobiopsy probe, in particular its working tip, and/or in order to prevent a freezing and/or adhesion of the biopsate to the recovery element. For example, the recovery element may be of plastic material, for example PTFE or silicone. Preferably, the surface of the recovery element (the first surface and/or the second surface) has an anti-adhesion coating acting against the adhesion of cells and/or tissue.

FIG. 2i shows that the recovery element is folded over even after it has been pulled through the stomach wall, after the recovery element and the working tip have left the opening and shields the biopsate in radial direction. In particular when the recovery element encloses the biopsate circumferentially enclosed, the biopsate is contaminated neither with stomach wall cells or germs from the inside stomach wall, nor has the biopsate been in contact with the stomach inside wall while the biopsate is being pulled through the opening, so that cells, in particular degenerated cells, or germs from the biopsate are not left in the stomach wall.

The folded-over recovery element 17 can also protect the biopsate 20 from unwanted stripping on the distal end 37 of the endoscope 36 or in the working channel 35 of the endoscope when the working tip 13 with the biopsate 20 is pulled back into the working channel 35 of the endoscope 36, for example, in order to recover the biopsate 20 through the working channel 35.

The recovery element can completely enclose the biopsate, i.e., not only circumferentially, but the folded-over recovery element, optionally together with the instrument, can enclose the biopsate on all sides. In particular, a region of the part of the folded-over retraction element for enclosing the biopsate can also be arranged distally between the distal end of the working tip and the environment. To do so, the recovery element is preferably long enough that the folded-over recovery element reaches beyond the distal end of the working tip and/or beyond the distal end of the biopsate. When the recovery element is being retracted, in particular the subsection of the part of the retraction element for enclosing the biopsate, said subsection projecting distally over the working tip and/or the biopsate, can be squeezed together, and opposite sections of the first surface can be brought into abutment and be held together by adhesive forces. This is shown, for example, by FIG. 2i′: The free end 27 of the recovery element 17 may be disposed to be bunched up and be held together by the adhesive forces—while being pulled through the opening 46—by the elastically deformed tissue of the stomach wall 41 that surrounds the opening 46, in order to extensively close the end 27. The biopsate 20 may be completely sheathed in this manner, i.e., also be separated from the environment in distal direction (in advance direction VR) by means of the recovery element 17. When the biopsate 20 is completely sheathed by the folded-over recovery element 17, a germ transfer can be prevented in a particularly effective manner.

The fastening section 18 may be glued or clamped, for example, to the shaft of the instrument 10. The recovery element 17 is preferably fastened to the instrument 10 in a fluid-tight manner. Adjoining the fastening section 18, there is provided—at a connecting site 50—the part 19 of the recovery element 17 which is folded over and which radially shields the biopsate 20. In the exemplary embodiment according to FIG. 1, the connecting site 50 is arranged proximal to the fastening section 18. In the embodiment according to FIG. 3, the connecting site 50 is arranged distally to the fastening section 18. The fixation of the recovery element 17 that has been changed in accordance with FIG. 3 can improve the folding-over properties of the recovery element 17 because the part of the recovery element 17 for shielding the biopsate 20 need not be pulled over the fastening section 18 during the folding-over procedure. FIG. 3b shows the assembly position or the folded-over orientation of the recovery element due to being pulled through the opening 46 in the organ wall 417. The recovery element may have the form of a funnel, in which case the funnel neck may form the fastening section 18. For assembly, the recovery element 17 may be pulled or pushed on the shaft 11, in which case the shaft 11 passes through the funnel neck. FIG. 3a shows the orientation for puncturing the organ wall 41 (see, for example, FIGS. 2a to 2i) in order to reach the site where the biopsate is to be removed. Initially, the recovery element 17 can be mounted in the assembly position or orientation (FIG. 3b) and then folded over into the orientation for puncturing, as shown by FIG. 3a. While the recovery element is being retracted through the opening 46 after receiving the tissue sample, the recovery element 17 is folded back in distal direction VR over the working tip 13, as is shown by FIG. 3b. The recovery element 17 can be folded over in distal direction VR, as well as in proximal direction ZR (for example, this also applies to the recovery element 17 according to FIGS. 1a, 1b).

FIG. 3c shows a diagram of a modification of the embodiment according to FIGS. 3a, b, as it may also occur in the remaining described embodiments. Corresponding to the modification according to FIG. 3c, the part 19 of the recovery element 17 for enclosing the biopsate that is to be harvested has a structure 58 on its first surface 25 that is initially oriented outward and that is turned inward during extraction of the biopsate by folding-over the recovery element 17. In FIG. 3d the instrument 10 is in a position that, on the one hand, corresponds to the position of the instrument 10 in FIG. 2d because the working tip 13 is located between the stomach wall 41 and the pancreas 45 and the part 19 of the recovery element is arranged for enclosing a biopsate in the opening 46. On the other hand, the working tip 13 is pressed against the tissue of the pancreas 45 or, as also shown by FIG. 2e, punctured into the pancreas 45. When the instrument 10 is retracted from the described position shown in FIG. 3d—without being able to push the instrument 10 first farther into advance direction VR so that the part 19 of the recovery element 17 is located between the stomach wall 41 and the pancreas 45, the structure 58 comes into positive and/or frictional engagement with the stomach wall. As a result of this, a folding-over of the retraction element 17 during retraction in recovery direction ZR is promoted. For example, the structure 58 may be a rough surface and/or the structure 58 may be made of at least one or more structuring elements such as, for example, scales that may form barbs, and/or nubs. The structure 58 offers an advantage in particular when the target area where the biopsate is to be harvested is so close behind the stomach wall 41 or another organ wall that the working tip 13 is already located in or on the tissue of the pancreas 45 or another organ while the part 19 of the recovery element 17 for enclosing is in a position according to FIG. 3d, i.e., not yet completely through the opening 46. In such a case it is possible that the end 27 of the recovery element 17 does not yet reach between the stomach wall 41 and the pancreas 45 and that the end 27 of the recovery element 17—during the retraction—is thus not in engagement with the stomach wall 41 or in abutment with the stomach wall surface that faces the pancreas 45.

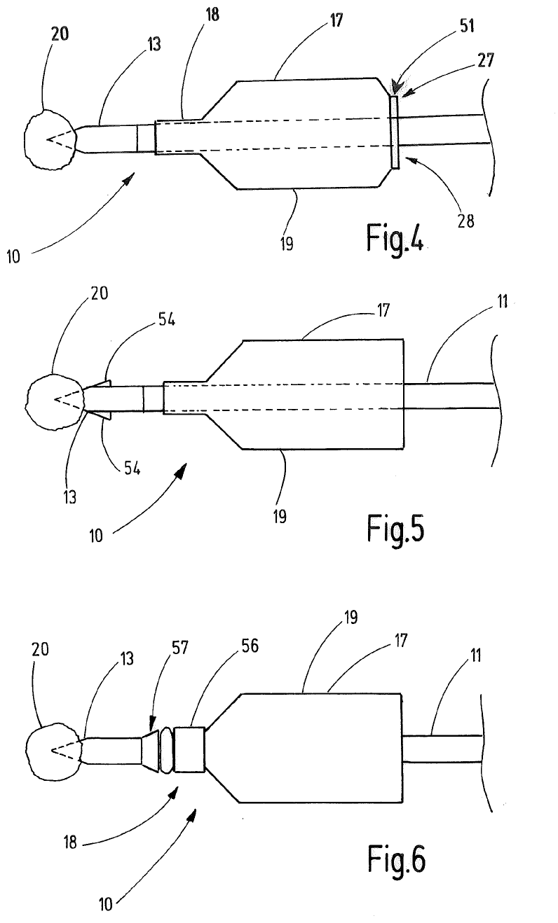

FIG. 4 shows another exemplary embodiment of an instrument 10 according to the invention. At the free end of the folding-over section of the recovery element, there is formed an elastic, for example circular or polygonal, ring 51, that forms a support element. The ring 51 has a greater inside diameter than the opening 46 in the organ wall 41 (see also FIGS. 2a to 2i, which show the formation of the opening 46 in the organ wall 41 and the harvesting of the biopsate 20 through the opening 46). While the instrument 10 is being slid through the opening 46 in order to harvest a biopsate 20, the ring 51 is squeezed together in order to fit in the opening 46. When the ring 51 has left the opening 46 on the other side of the organ wall 46, the ring 51 relaxes again due to its elasticity, and thus tries—due to the elasticity and upon the absence of the external force outside the opening 46—to again assume a greater diameter transverse relative to the advance direction VR, and/or its original form. During retraction, the recovery element 17 supports itself by means of the ring 51 around the opening 46 at a radial distance from the opening 46 against the organ wall 41 and, in doing so, promotes the folding-over of the recovery element 17. Preferably, the support element 51 is arranged on the end 27 of the recovery element 17 in such a manner that the support element 51—while the instrument 10 is being retracted through the opening 46 with the harvested biopsate 20—first passes through the opening 46 in the organ wall 41 after the biopsate—shielded by means of the recovery element 17—has entered the opening 46 or left the opening 46 in the organ wall 41. Preferably, the support element 51 is disposed to support itself during retraction against the organ wall 46 preferably until the recovery element 17 has been folded over.

As shown by FIG. 4, the diameter of the opening 28 on the end 27 of the recovery element 17, which opening 28 may be formed by the ring 51 as in the depicted exemplary embodiment, can preferably be smaller than the inside diameter of the recovery chamber 53.

FIG. 5 shows an embodiment of the instrument 10 according to the invention that comprises cutting elements 54 arranged on the working tip 13, said cutting elements improving passage through the organ wall to the site where the biopsate 20 is to be removed.

FIG. 6 shows another exemplary embodiment of an instrument 10 according to the invention, in which the fastening section 18 is enclosed by means of a fastening ring 56 and clamped (crimped) to the shaft 11. In order to adapt the transition from the shaft diameter or the diameter of the working tip to the diameter of the fastening section 18 or the fastening ring 56, there is arranged—between the working tip 13 and the fastening section 18—a form element 57 having an outside diameter that widens in the direction of the fastening section 18, for example a conically widening outside diameter. This form element 57 facilitates the guiding of the instrument 10 through the opening in the organ wall—at least in puncturing direction.

Whereas the biopsy instrument preferably is a biopsy probe, the biopsate recovery device can also be used with other biopsy probes in order to separate a biopsate from the environment while it is being removed, in order to protect the environment or the biopsate, or to prevent a loss of the biopsate. For example, the biopsate recovery device can also be used on a biopsy probe with which a biopsate is punched out of the tissue.

Whereas the recovery element is essentially made as a one-piece part or flexible sheath, in particular as a seamless one-piece part, the recovery element may also consist of several parts which are connected to each other so as to be movable relative to another to form a mechanical element. For example, the recovery element may be designed similarly to a folding bellows. In particular considering embodiments in which the recovery element is a one-piece part, the dimensioning of the recovery element and/or different materials for the different sections of the recovery element can affect the stiffness of the different sections of the recovery element in a targeted manner.

FIG. 7 shows an exemplary embodiment of a biopsy instrument 10 comprising a working tip 13 with a region 60 that is disposed for adhering to the biopsate. The region 60 has a greatest diameter d2 that is smaller than the diameter d3 of the shaft 11—measured at a location at or in the recovery element 17. This allows an exemplary embodiment, wherein the ratio of the diameter d3 of the shaft 11—measured at a location at or in the recovery element 17—is 1:1 to the greatest outside diameter d4 of the recovery element. In the depicted exemplary embodiment, the ratio is greater than 1:1. The ratio is preferably at most 1:1 to at least 1:6, particularly preferably at most 1:3 to at least 1:5.

With reference to a further exemplary embodiment, FIG. 8 shows that the length 1 of the section of the instrument 10 which is disposed for receiving—in particular by adhesion—a biopsate 20 and along which extends the part 19 of the recovery element 17 while enclosing the biopsate 20 and thus separates the section, preferably corresponds to 4 to 10 times the diameter d3 of the shaft 11—measured at a location at or in the recovery element 17. FIG. 8 illustrates a recovery element that encloses a biopsate outside the body of the patient, where no forces act from the outside on the recovery element, which forces could compress the recovery element. Section 31 of the recovery element 17 for enclosing the biopsate 20 may be resilient in such a manner that the section 31, when the section 31 is pressed against the biopsate 20 while the working tip 13 is being pulled out of the body of the patient, adapts to the biopsate 20 and remains in intimate contact with the biopsate 20 when the working tip 13 has been pulled out of the body and, thus the external force due to the resistance of the patient tissue against deformation is eliminated, said force pressing the section 31 against the biopsate 20 while it is being pulled out.

The material or materials of the recovery element in the exemplary embodiments may display hardnesses of at least 20 to at most 80 Shore A, preferably at least 40 to at most 60 Shore A. In the exemplary embodiments, the recovery element may be composed of materials such as, e.g., different silicones, that display different hardnesses. The hardness may decrease in longitudinal direction L of the recovery element in the direction toward the free end 27 of the recovery element 17, for example be greater in the connecting section 30 than in the section 31 for enclosing the biopsate 20. FIG. 8 shows an exemplary embodiment of a recovery element which displays a harder material in the fastening section of the recovery element, for example, than in the connecting section 30 of the recovery element 18 the adjoins in the direction toward the free end of the recovery element 17, said connecting section 30 connecting the section 31 of the recovery element 17 for enclosing the biopsate 20 to the fastening section 18. Alternatively or additionally, the material of the connecting section 30 may be harder than the material of section 31 of the recovery element 17 for enclosing the biopsate 20. The hardness difference between the materials may be at least 5 Shore A, for example. For example, in the exemplary embodiments the hardness of the sections may increase linearly, progressively or degressively from the section 31 for enclosing being the least hard section, via the connecting section 30, to the fastening section 18 being the hardest of the three sections 18, 30, 31.

With reference to an exemplary embodiment, FIG. 9 shows that embodiments of the recovery element 17 may comprise an opening up structure 61. The recovery element 17 may comprise an opening up structure 61 which has—extending away from a base section or base element 62—elongated elements and/or sections 63 at a distance from each other in circumferential direction U, which—due to the stiffness of the connections of the base section or base element 62—attempt, with the elements and/or sections 63 extending away from the latter, to resume their original orientation relative to the base section or base element 62 upon elimination of the external force and to thus open up the mounting structure 61, after, for example, the base section and/or the base element 62 has been moved through a narrow opening 46 of a tissue wall into another region. The elongated elements or sections 63 that are spaced apart in circumferential direction ensure that the subsection 30 can be radially compressed in a controlled manner when the subsection 30 is pushed through an opening 46 in a tissue wall, and the connection to the base section or base element 62 ensures that the subsection 30, due to the elastic resetting forces will again open particularly reliably in order to resume its original form. FIGS. 9a and 9b show that the recovery element 17 has elongated sections 63, for example in the form of a wall thickness that alternatingly increases and decreases in circumferential direction and/or may have elongated reinforcing element sections 63 that may consist of another material than the subsection 30, e.g., of metal or another polymer. The regions displaying different wall thicknesses may be originally formed together of the same material, and/or a base material of the recovery element 17 is coated with the same or a different material for thickening. The elongated sections 63 may extent into the section 31 for enclosing the biopsate or end in the connecting section 30 in front of the section 31.

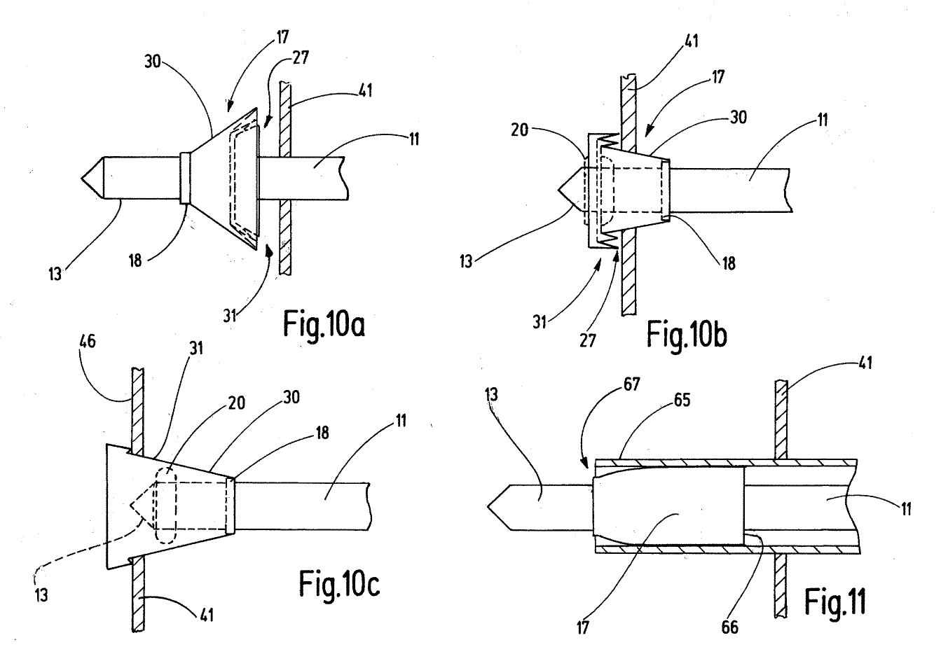

In the embodiments, the section 31 of the recovery element 17 for enclosing the biopsate 20 can—for the puncturing by the biopsy instrument—be received in the connecting section 30 of the recovery element 17 for positioning said instrument. Preferably, the section 30 for enclosing the biopsate 20 can be rolled and/or folded into the connecting section 30. FIG. 10a shows an example of a biopsy instrument 10 after puncturing an organ wall 41 for positioning. The free end 27 of the recovery element or the opening in the connecting section 30 faces away from the distal end of the working tip 13. The section 31 of the recovery element 17 on the free end 27 is folded into the connecting section 30. As a result of this, it is prevented that the recovery element 17—during puncture or insertion—is pulled too far lengthwise in proximal direction. The folded-in and/or rolled-in region forms a ring displaying a relatively great resistance to radial deformation, which promotes opening up after puncturing the wall 41. When the biopsy instrument 10 is retracted back through the wall 41 in order to remove the sample 20 (FIG. 10b), the recovery element is essentially folded over up to the fastening section, and the ring which has formed by folding-in and/or rolling-in is moved toward the outside. During retraction, the section 31 for enclosing the biopsate 20 is automatically unfolded and, in doing so, pulled over the biopsate (FIG. 10c).

Biopsy instruments 10 according to the invention may comprise a securing element 68 which is disposed to prevent an unwanted folding-over of the recovery element 17 while the working tip 13 is being positioned for severing the biopsate 20. For retracting the biopsy instrument 10 for removal of the biopsate, it is possible to deactivate the securing element 65, so that a folding-over is possible during retraction. The securing element 65 can be pulled off the recovery element 17 in order to allow the folding-over. Preferably, the securing element 65 encloses a proximally facing edge 66 of the recovery element 17, which edge may be formed by the free end 27 or, for example, by a ring formed by rolling-in and/or folding-in, as described in conjunction with the exemplary embodiment according to FIGS. 10a to 10c. The reason being that—during retraction—the edge 66 is disposed to come into engagement with the tissue wall 41 in such a manner that the recovery element 17 is folded over. The securing element 65 may be connected to the biopsy instrument 10 and/or the recovery element 17, in which case the connection 67 must be released in order to eliminate securing. To do so, the connection can be set up to require destruction when released. The connection can be set up in such a manner that, during contact with fluid, in particular tissue fluid, it is dissolved or at least weakened, so that the securing element 65 can be released from the recovery element 17 or the biopsy instrument 10. FIG. 11 shows an exemplary embodiment of a securing element 65 in the form of a sheath tube. For example, the sheath tube 65 may be selectively connected to the recovery element 17 with a connection 67 that can be destroyed with minimal force or may loosely enclose the recovery element 17. The length of the sheath tube 65 is preferably such that the sheath tube 65—with the working tip 13 in position—extends at least beyond the edge 66 of the recovery element 17 or even at least through the opening 46 in the tissue wall 41. The securing element 65 is set up so that the user—upon reaching the end position of the biopsy instrument 10 (position in which the biopsate is removed) and before retracting the working tip 13 with the biopsate—can retract the sheath tube 65 in proximal direction of the recovery element 17, optionally while destroying a connection 67 of the securing element 65 with the recovery element 65 or another section of the biopsy instrument 10.

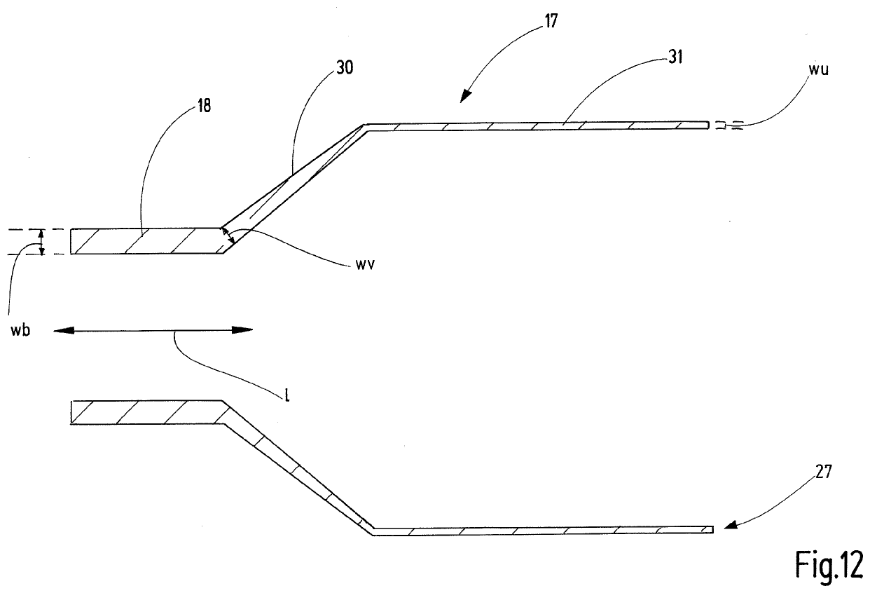

With reference to an exemplary embodiment, a recovery element 17 according to the invention having a wall thickness decreasing in the direction toward the free end 27 of the recovery element is shown by FIG. 12. The greatest wall thickness wv of the connecting section 30 may be greater than the greatest wall thickness wu of the section 31 for enclosing the biopsate. The greatest wall thickness wb of the fastening section 18 may be greater than the greatest wall thickness wv of the connecting section 30. In the embodiments as described in conjunction with FIGS. 9a, 9b, the greatest wall thickness wv of an elongated section 63 or the greatest wall thickness between two elongated sections 63 may be greater than the greatest wall thickness 2b of the section 31 for enclosing the biopsate and/or smaller than the greatest wall thickness wb of the fastening section 18. It is possible to measure the greatest wall thickness wu—when the sections 63 of the opening up structure 61 extend into the section 31 for enclosing the biopsate 20—outside the elongated sections 63, for example. The wall thickness of the recovery element may continuously (steadily) decrease from a section to a section adjoining in the direction toward the free end 27—for example, from the connecting section 30 to the section 31 for enclosing. For example, the wall thickness may decrease linearly in the direction toward the free end 27. Instead of the greatest wall thickness, it is possible to compare the wall thicknesses that have been averaged over the lengths of the individual sections, in which case the average wall thickness may decrease in the direction toward the free end 27 of the recovery element 17, for example.

The features of the embodiments described herein may be combined with each other as desired. For example, features described in conjunction with FIG. 12 can be combined with features that relate to the hardness of different materials, the adaptability of the section 31 to enclose the biopsate 20, the diameter ratios at the ends of the connecting section 30, an opening up structure 61 and/or the ability of section 31 for enclosing to roll in or fold in the connecting section 30 in embodiments.

Features such as different wall thickness, material displaying different hardnesses and/or a mounting structure 65 are examples of features which can be used to form embodiments that display a stiffness of the recovery element against radial deformation decreasing toward the free end 27. For example, the connecting section 30 may be stiffer against radial deformation than the section 31 for enclosing the biopsate 20, at least when it is rolled out and/or unfolded.

A biopsate recovery device 16 for recovering a biopsate 20 through an opening 46 with a recovery element 17 for fastening to or adjacent to a working tip 13 of a biopsy instrument 10 is disclosed, wherein the recovery element 17 is set up to—while the working tip 13 is being retracted through the opening 46—be folded over the biopsate 20 in order to enclose the biopsate during the retraction of the working tip 13 through the opening 46 in order to separate the biopsate 20 from the environment during retraction through the opening 46. As a result of this, the biopsate 20 and/or the environment can be protected against contamination and/or cell transfer and/or germ transfer, and/or the biopsate 20 can be protected against being inadvertently stripped off at the opening 46.

### LIST OF REFERENCE SIGNS

| | |
|---|---|
| 10 | Biopsy instrument/instrument |
| 11 | shaft |
| 12 | Distal end of the shaft |
| 13 | Working tip |
| 14 | Plastic sheath |
| 15 | Distal end of the instrument |
| 16 | Biopsate recovery device |
| 17 | Recovery element |
| 18 | Fastening section |
| 19 | Part of the recovery element for enclosing the biopsate |
| 20 | Biopsate |
| 25 | First surface |
| 26 | Second surface |
| 27 | End |
| 28 | Opening |
| 29 | Recovery chamber |
| 30 | Subsection/connecting section |
| 31 | Additional subsection/section for enclosing the biopsate |
| 35 | Working channel |
| 36 | Endoscope |
| 37 | Distal End of the endoscope |
| 40 | Stomach |
| 41 | Stomach wall |
| 45 | Pancreas |
| 46 | Opening |
| 47 | Structure |
| 50 | Connecting site |
| 51 | Ring/support element |
| 53 | Inside diameter, recovery chamber |
| 54 | Cutting elements |
| 56 | Fastening ring |
| 57 | Form element |
| 58 | Structure |
| 60 | Region |
| 61 | opening up structure |
| 62 | Base section or base element |
| 63 | Elongated element or section |
| 65 | Securing element |
| 66 | Edge |
| 67 | Connection |
| D1 | Inside diameter, recovery chamber |
| VR | Puncturing direction/advance direction/distal direction |
| ZR | Retracting direction/recovery direction/proximal direction |
| P | Arrows |
| da1 | Outside diameter |
| da2 | Outside diameter |
| d2 | Diameter of region 60 |
| d3 | Diameter of shaft 11 |
| d4 | Outside diameter of the recovery element 17 |
| l | Length |
| U | Circumferential direction |
| wb | Wall thickness |
| wv | Wall thickness |
| Wu | Wall thickness |
| L | Longitudinal direction |

1. A biopsate recovery device (16) for recovering a biopsate (20) through an opening (46) in an organ or tissue wall or an instrument, the biopsate recovery device comprising:
a recovery element (17) configured to be positioned at or adjacent to a working tip (13) of a biopsy instrument (10), wherein the recovery element (17) is configured to be folded over the biopsate (20) harvested by the working tip while the working tip (13) is being retracted through the opening (46) in order to enclose the biopsate (20) within the recovery element so as to separate the biopsate (20) from a surrounding environment during the retraction through the opening (46).

2. The biopsate recovery device (16) according to claim 1, wherein the recovery element (17) is configured to enclose the biopsate (20) in a circumferentially closed manner in order to fluidically separate the biopsate (20) from the surrounding environment.

3. The biopsate recovery device (16) according to claim 1, wherein the recovery element (17) includes a wall engaging portion that is configured to come into engagement with the organ or tissue wall around the opening while the working tip is being retracted in order to promote the folding-over of the recovery element (17) over the biopsate while the biopsy instrument (10) is being retracted through the opening (46).

4. The biopsate recovery device (16) according to claim 1, wherein the recovery element (17) comprises a support element (51) that is configured to be elastically deformed in a direction transverse to a retracting direction (ZR) and is configured to engage the organ or tissue wall (41) surrounding the opening (46) during retraction.

5. The biopsate recovery device (16) according to claim 1, wherein the recovery element (17) is formed of a material or has a coating configured to prevent the recovery element (17) from freezing to the biopsy instrument (10) and/or the biopsate (20) from freezing to the recovery element (17).)

6. The biopsate recovery device (16) according to claim 1, wherein the recovery element (17) has, adjoining or adjacent to a fastening site of the recovery element (17) that connects the recovery element to the biopsy instrument (10), a section (30) having an outside diameter that widens along a longitudinal section of the biopsy instrument (10).)

7. The biopsate recovery device (16) according to claim 1, wherein the recovery element (17) has an unexpanded length and is configured to elastically expand to at most five times its unexpanded length, while passing through the organ or tissue wall (41).

8. The biopsate recovery device (16) according to claim 1, wherein a wall of the recovery element (17) has a thickness from 0.005 mm to 2 mm.

9. The biopsate recovery device (16) according to claim 1, wherein the recovery element (17) has, in a longitudinal direction (L) thereof sequential sections (18, 30, 31) (17), wherein each of the sequential sections is formed of a different material than the other sequential section or sections immediately adjacent to said sequential section.

10. The biopsate recovery device (16) according to claim 1, wherein the recovery element (17) has a surface which inhibits adhesion of cells and/or tissue.

11. A biopsy instrument (10) with the biopsate recovery device (16) according to claim 1.

12. The biopsy instrument (10) according to claim 11, wherein the biopsy instrument (10) is a cryobiopsy probe.

13. The biopsy instrument (10) according to claim 11, wherein the biopsy instrument comprises a working tip (13) configured for puncturing an organ wall (41).

14. The biopsy instrument (10) according to claim 11, wherein the recovery element (17) has a section (31) configured to enclose the biopsate (20), wherein the section (31) for enclosing the biopsate (20) is configured to be received in a section (30) of the recovery element (17) that connects the recovery element to the biopsy instrument.

15. The biopsy instrument (10) according to claim 11, further comprising a securing element (65) configured to securing keep the recovery element (17) from folding over.

16. The biopsate recovery device according to claim 1, wherein the recovery element (17) has a fastening section (18), a section (31) for enclosing the biopsate, and a connecting section (30) that connects the section (31) for enclosing the biopsate (20) to the fastening section (18), wherein each section has a wall thickness (wu), and the wall thickness of the section (31) for enclosing the biopsate (20) is less than the wall thickness (wv) of the connecting section (30) measured at a thickest portion of the connecting section.
